(19) United States
(12) Patent Application Publication
(10) Pub. No.: US 2007/0128291 A1
(43) Pub. Date: Jun. 7, 2007

(54) Method and Apparatus for Forming Chromonic Nanoparticles

(76) Inventors: Jeffrey H. Tokie, Scandia, MN (US); Hassan Sahouani, Hastings, MN (US); Jacqueline M. Aguilera, Chisago City, MN (US)

Correspondence Address: 3M INNOVATIVE PROPERTIES COMPANY, PO BOX 33427, ST. PAUL, MN, 55133-3427, US

(21) Appl. No.: 11/275,070

(22) Filed: Dec. 7, 2005

(51) Int. Cl. A61K 38/28; A61M 11/00; A61K 9/50; A61K 9/16

(52) U.S. Cl. 424/490; 514/3; 128/200.23

(57) ABSTRACT

A method and apparatus for forming guest molecules encapsulated with chromonic material are described. The method includes atomizing a solution to form a pre-atomized particle stream. The solution includes chromonic material, a guest molecule and a carrier fluid. Then, atomizing the pre-atomized particle stream to form an atomized particle stream; evaporating at least a portion of the carrier fluid from the atomized particle stream to form a dried atomized particle stream, and forming encapsulated guest molecules from the dried atomized particle stream.

### BACKGROUND

The present disclosure relates to the field of chromonics. In particular, the present disclosure relates to methods and apparatus useful for forming chromonic nanoparticles useful for encapsulating and controlled release of guest molecules, such as drugs.

Encapsulation and controlled release of a substance or material may be achieved by a number of methods. Typically, a polymeric coating may be used to either surround a substance or to form a mixture with a substance. Another common approach uses macroscopic structures having openings or membranes that allow for release of a substance. Encapsulation and controlled release finds broad utility, but is particularly useful in the field of controlled release drug delivery.

Many polymeric coatings operate to control release by swelling in the presence of water. This relies on the mechanism of diffusion through a swollen matrix, which can be difficult to control. Alternatively polymeric coatings or mixtures of polymers with a substance may also operate through erosion or degradation of the polymer. In either case, it can be difficult to control the release rate, since most polymers are highly polydisperse in nature. In addition, there are a limited number of polymers suitable for use in pharmaceutical applications, and a given polymer may interact with different substances in very different and unpredictable ways.

Macroscopic structures, such as osmotic pumps, control release by uptake of water from the environment into a chamber containing a substance that is forced from the chamber through a delivery orifice. This, however, requires a complex structure that needs to be prepared and filled with the substance that is to be delivered.

Protection of a drug from adverse environmental conditions may be desirable in certain drug delivery applications. The gastrointestinal tract represents one example of an environment that can interfere with the therapeutic efficacy of a drug. The ability to selectively protect a drug from certain environmental conditions, such as the low pH of the stomach, and to also be able to selectively and controllably deliver the drug under other environmental conditions, such as the neutral pH of the small intestine, is highly desirable.

Encapsulation of some molecules with chromonics has been described. Encapsulation is best when the encapsulation solution has a chromonic concentration greater than one or two percent. However, it is difficult to generate sub-micrometer particles using encapsulation solutions having a chromonic concentration greater than one or two percent.

### SUMMARY

The present disclosure provides methods and apparatus useful for forming chromonic nanoparticles. In many embodiments, these chromonic nanoparticles are useful for encapsulating and controlled release of guest molecules, such as drugs.

In one aspect, a method for forming guest molecules encapsulated with chromonic material is described. The method includes atomizing a solution to form a pre-atomized particle stream. The solution includes chromonic material, a guest molecule and a carrier fluid. Then, atomizing the pre-atomized particle stream to form an atomized particle stream, evaporating at least a portion of the carrier fluid from the atomized particle stream to form a concentrated atomized particle stream, and forming encapsulated guest molecules from the concentrated atomized particle stream.

In another aspect, an apparatus for forming guest molecules encapsulated with chromonic material is described. The apparatus includes a solution reservoir in fluid communication with a first atomizer. The solution reservoir contains chromonic material, a guest molecule and a carrier fluid. A second atomizer is in fluid communication with the first atomizer, and a drying chamber in fluid communication with the second atomizer.

The above summary is not intended to describe each disclosed embodiment or every implementation of the present disclosure. The Figures, Detailed Description and Examples that follow more particularly exemplify these embodiments.

### BRIEF DESCRIPTION OF THE DRAWINGS

The disclosure may be more completely understood in consideration of the following detailed description of various embodiments in connection with the accompanying drawings, in which:

FIG. 1 is a schematic diagram of an illustrative apparatus for forming the encapsulated guest molecules using chromonic nanoparticles.

While the invention is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the invention.

### DETAILED DESCRIPTION

The present disclosure provides methods and apparatus useful for forming chromonic nanoparticles. In many embodiments, these chromonic nanoparticles are useful for encapsulating and controlled release of guest molecules, such as drugs.

For the following defined terms, these definitions shall be applied, unless a different definition is given in the claims or elsewhere in this specification.

The term, “chromonic materials” (or “chromonic compounds”) refers to large, multi-ring molecules typically characterized by the presence of a hydrophobic core surrounded by various hydrophilic groups (see, for example, Attwood, T. K., and Lydon, J. E., Molec. Crystals Liq. Crystals, 108, 349 (1984)). The hydrophobic core can contain aromatic and/or non-aromatic rings. When in solution, these chromonic materials tend to aggregate into a nematic ordering characterized by a long-range order.

The recitation of numerical ranges by endpoints includes all numbers subsumed within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. Thus, for example, reference to a composition containing “a compound” includes a mixture of two or more compounds. As used in this specification and the appended claims, the term “or” is generally employed in its sense including “and/or” unless the content clearly dictates otherwise.

Unless otherwise indicated, all numbers expressing quantities of ingredients, measurement of properties and so forth used in the specification and claims are to be understood as being modified in all instances by the term “about.” Accordingly, unless indicated to the contrary, the numerical parameters set forth in the foregoing specification and attached claims are approximations that can vary depending upon the desired properties sought to be obtained by those skilled in the art utilizing the teachings of the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviations found in their respective testing measurements.

Any chromonic material can be useful in the methods described herein. Compounds that form chromonic phases are known in the art, and include, for example, xanthoses (for example, azo dyes and cyanine dyes) and perylenes (see, for example, Kawasaki et al., Langmuir 16, 5409 (2000), or Lydon, J., Colloid and Interface Science, 8, 480 (2004)). Representative examples of useful chromonic materials include di- and mono-palladium organyls, sulfamoyl-substituted copper phthalocyanines, and hexaaryltryphenylene.

In many embodiments, the chromonic material or chromonic molecule is a non-polymeric molecule containing more than one carboxy functional group and can associate with multi-valent cations to form a water-insoluble matrix that is capable of encapsulating a guest molecule and that is further capable of subsequently controllably releasing the guest molecule. The carboxy groups may be directly attached to an aromatic or heteroaromatic functional group (e.g., carboxyphenyl). When the chromonic molecule has more than one aromatic or heteroaromatic functional group, the carboxy groups are arranged such that each aromatic or heteroaromatic group has no more than one carboxy group directly attached. Examples of such chromonic molecules include aurintricarboxylic acid, pamoic acid, 5-{4-[[4-(3-carboxy-4-chloroanilino)phenyl](chloro)phenylmethyl]anilino}-2-chlorobenzoic acid, aluminon ammonium salt, and triazine derivatives described in U.S. Pat. No. 5,948,487 (Sahouani, et al.), the disclosure of which is incorporated by reference.

In many embodiments, the chromonic molecule contains at least one formal positive charge. In another aspect, the chromonic molecule may be zwitterionic, that is, carrying at least one formal positive and one formal negative charge. Zwitterionic chromonic molecules will carry at least one negative charge. In one aspect, the negative charge will be carried through a carboxy group having a dissociated hydrogen atom, —COO⁻. The negative charge may be shared among the multiple carboxy functional groups present, such that a proper representation of the chromonic molecule consists of two or more resonance structures. Alternatively, the negative or partial negative charges may be carried by other acid groups in the chromonic molecule.

In many embodiments, the chromonic molecules include triazine derivatives with the structure below.

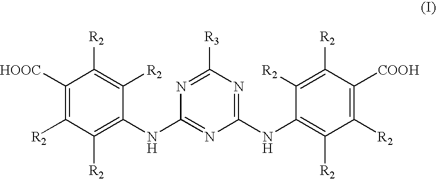

Formula I above shows an orientation of the carboxy (—COOH) group that is para with respect to the amino linkage to the triazine backbone of the compound. As depicted above the chromonic molecule is neutral, but it may exist in alternative forms, such as a zwitterion or proton tautomer, for example where a hydrogen atom is dissociated from one of the carboxyl groups and is associated with one of the nitrogen atoms in the triazine ring. The chromonic molecule may also be a salt. The carboxy group may also be meta with respect to the amino linkage, as shown in formula II below (or may be a combination of para and meta orientations, which is not shown).

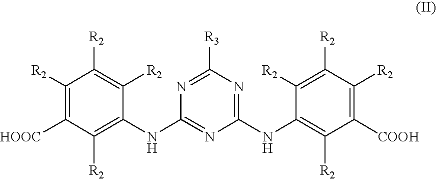

Each R₂ is independently selected from any electron donating group, electron withdrawing group and electron neutral group. In many embodiments, R₂ is hydrogen or a substituted or unsubstituted alkyl group. In some embodiments, R₂ is hydrogen, an unsubstituted alkyl group, or an alkyl group substituted with a hydroxy, ether, ester, sulfonate, or halide functional group. In one embodiment, R₂ is hydrogen.

R₃ may be selected from the group consisting of: substituted heteroaromatic rings, unsubstituted heteroaromatic rings, substituted heterocyclic rings, and unsubstituted heterocyclic rings, that are linked to the triazine group through a nitrogen atom within the ring of R₃. R₃ can be, but is not limited to, heteroaromatic rings derived from pyridine, pyridazine, pyrimidine, pyrazine, imidazole, oxazole, isoxazole, thiazole, oxadiazole, thiadiazole, pyrazole, triazole, triazine, quinoline, and isoquinoline. In many embodiments, R₃ comprises a heteroaromatic ring derived from pyridine or imidazole. A substituent for the heteroaromatic ring R₃ may be selected from, but is not limited to, any of the following substituted and unsubstituted groups: alkyl, carboxy, amino, alkoxy, thio, cyano, amide, sulfonate, hydroxy, halide, perfluoroalkyl, aryl, ether, and ester. In many embodiments, the substituent for R₃ is selected from alkyl, sulfonate, carboxy, halide, perfluoroalkyl, aryl, ether, and alkyl substituted with hydroxy, sulfonate, carboxy, halide, perfluoroalkyl, aryl, and ether. In one embodiment, R₃ is a substituted pyridine; the substituent being preferably located at the 4-position. In another embodiment, R₃ is a substituted imidazole; the substituent being preferably located at the 3-position. Suitable examples of R₃ include, but are not limited to: 4-(dimethylamino)pyridium-1-yl, 3-methylimidazolium-1-yl, 4-(pyrrolidin-1-yl)pyridium-1-yl, 4-isopropylpyridinium-1-yl, 4-[(2-hydroxyethyl)methylamino]pyridinium-1-yl, 4-(3-hydroxypropyl)pyridinium-1-yl, 4-methylpyridinium-1-yl, quinolinium-1-yl, 4-tert-butylpyridinium-1-yl, and 4-(2-sulfoethyl)pyridinium-1-yl, shown in formulae IV to XIII below. Examples of heterocyclic rings that R₃ may be selected from include, for example, morpholine, pyrrolidine, piperidine, and piperazine.

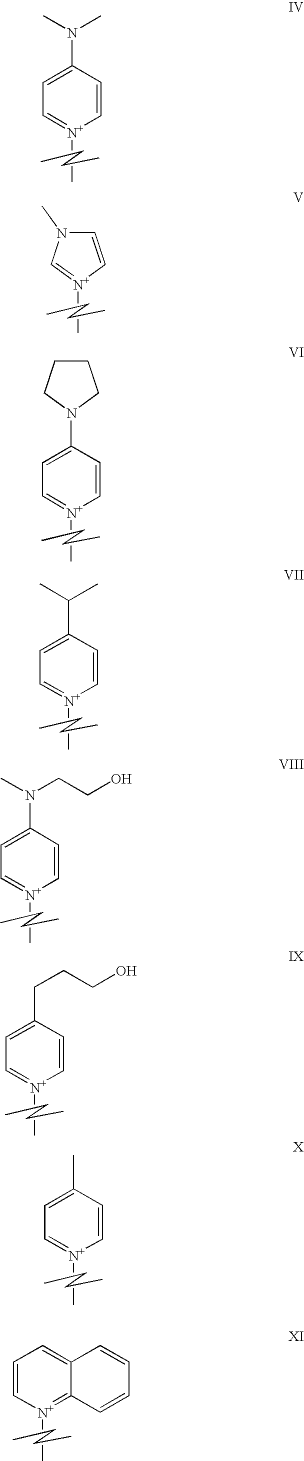

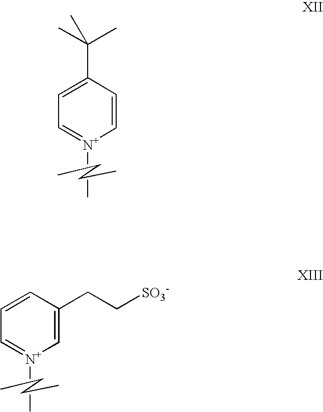

In one aspect, the R₃ group shown in formula V above may also have a substituent group other than methyl attached to the imidazole ring, as shown below, 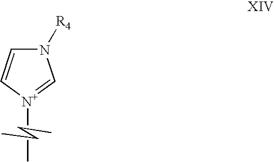
where R₄ is hydrogen or a substituted or unsubstituted alkyl group. In many embodiments, R₄ is hydrogen, an unsubstituted alkyl group, or an alkyl group substituted with a hydroxy, ether, ester, sulfonate, or halide functional group. In some embodiments, R₄ is propyl sulfonic acid, methyl, or oleyl.

As depicted above the chromonic molecule of formula I and II is neutral, however chromonic molecules described herein may exist in an ionic form wherein they contain at least one formal positive charge. In one embodiment, the chromonic molecule may be zwitterionic. An example of such a zwitterionic chromonic molecule, 4-{[4-(4-carboxyanilino)-6-(1-pyridiniumyl)-1,3,5-triazin-2-yl]amino}benzoate, is shown in formula III below where R₃ is a pyridine ring linked to the triazine group through the nitrogen atom of the pyridine ring. As shown, the pyridine nitrogen carries a positive charge and one of the carboxy functional groups carries a negative charge (and has a dissociated cation, such as a hydrogen atom), —COO⁻.

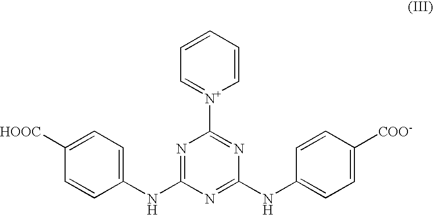

The chromonic molecule shown in formula III may also exist in other tautomeric forms, such as where both carboxy functional groups carry a negative charge and where positive charges are carried by one of the nitrogens in the triazine group and the nitrogen on the pyridine group.

As described in U.S. Pat. No. 5,948,487 (Sahouani, et al.), triazine derivatives with formula I may be prepared as aqueous solutions, or may be prepared as salts which can later be re-dissolved to form an aqueous solution. A typical synthetic route for the triazine molecules shown in I above involves a two-step process. Cyanuric chloride is treated with 4-aminobenzoic acid to give 4-{[4-(4-carboxyanilino)-6-chloro-1,3,5-triazin-2-yl]amino}benzoic acid. This intermediate is treated with a substituted or unsubstituted nitrogen-containing heterocycle. The nitrogen atom of the heterocycle displaces the chlorine atom on the triazine to form the corresponding chloride salt. The zwitterionic derivative, such as that shown in formula III above, is prepared by dissolving the chloride salt in ammonium hydroxide and passing it down an anion exchange column to replace the chloride with hydroxide, followed by solvent removal. Alternative structures, such as that shown in II above, may be obtained by using 3-aminobenzoic acid instead of 4-aminobenzoic acid.

These molecules that are non-covalently crosslinked are capable of forming a chromonic phase before they are in the presence of multi-valent cations (i.e., before they are crosslinked). Chromonic phases or assemblies are known and consist of stacks of flat, multi-ring aromatic molecules. The molecules consist of a hydrophobic core surrounded by hydrophilic groups. The stacking takes on a number of morphologies, but is typically characterized by a tendency to form columns created by a stack of layers. Ordered stacks of molecules are formed that grow with increasing concentration, but they are distinct from micellar phases, in that they generally do not have surfactant-like properties and do not exhibit a critical micellar concentration. In many embodiments, the chromonic phases will exhibit isodesmic behavior, that is, addition of molecules to the ordered stack leads to a monotonic decrease in free energy. In one aspect, the molecules that are non-covalently crosslinked are host molecules that will form either a chromonic M or N phase in aqueous solution before they are in the presence of multi-valent cations (i.e., before they are crosslinked). In another aspect, the molecules that are non-covalently crosslinked are chromonic molecules that will form either a chromonic M or N phase in an alkaline aqueous solution before they are in the presence of multi-valent cations (i.e., before they are crosslinked). The chromonic M phase typically is characterized by ordered stacks of molecules arranged in a hexagonal lattice. The chromonic N phase is characterized by a nematic array of columns, that is, there is long range ordering along the columns characteristic of a nematic phase, but there is little or no ordering amongst the columns, thus it is less ordered than the M phase. The chromonic N phase typically exhibits a schlieren texture, which is characterized by regions of varying index of refraction in a transparent medium.

These chromatic molecules can be formed into chromonic nanoparticles that are useful for encapsulating and controlled release of guest molecules, such as drugs. These chromatic nanoparticles can form a water-insoluble matrix that is non-covalently crosslinked by multi-valent cations. This crosslinking forms a three-dimensional matrix that is insoluble in water. By non-covalent, it is meant that the crosslinking does not involve permanently formed covalent (or chemical) bonds. That is, the crosslinking does not result from a chemical reaction that leads to a new, larger molecule, but rather results from associations of the cations with the chromatic molecules that are strong enough to hold them together without undergoing a chemical reaction. These interactions are typically ionic in nature and can result from interaction of a formal negative charge on the chromatic molecule with the formal positive charge of a multi-valent cation. Since the multi-valent cation has at least two positive charges, it is able to form an ionic bond with two or more chromatic molecules, that is, a crosslink between two or more chromatic molecules. The crosslinked, water-insoluble matrix arises from the combination of direct chromatic molecule-chromatic molecule interactions and chromatic molecule-cation interactions. Divalent and/or trivalent cations are preferred. In some embodiments, a majority of the multivalent cations are divalent. Suitable cations include any divalent or trivalent cations, with calcium, magnesium, zinc, aluminum, and iron.

In one aspect where the chromatic molecules form a chromonic phase or assembly in an aqueous solution, the chromatic molecules may form columns created from layered stacks of chromatic molecules. The multi-valent cations provide crosslinks between these columns. Although not wishing to be bound by any particular theory, it is believed that the chromatic molecules associate with each other through interaction of the aromatic functionality and the carboxy functionality. Alternatively, a multi-valent cation may associate with two or more chromatic molecules, which in the case of a divalent cation forms a “dimer” that precipitates from solution and the precipitated “dimers” interact with each other through the host molecule functionality to form a water-insoluble matrix.

The chromonic composition is characterized in that a guest molecule may be encapsulated and released by the chromonic nanoparticles. Examples of useful guest molecules include dyes, cosmetic agents, fragrances, flavoring agents, and bioactive compounds, such as drugs, herbicides, pesticides, pheromones, and antifungal agents. A bioactive compound is herein defined as a compound intended for use in the diagnosis, cure, mitigation, treatment or prevention of disease, or to affect the structure or function of a living organism. Drugs (i.e., pharmaceutically active ingredients) are particularly useful guest molecules, which are intended to have a therapeutic effect on an organism. Alternatively, herbicides and pesticides are examples of bioactive compounds intended to have a negative effect on a living organism, such as a plant or pest. Although any type of drug may be employed with compositions of the present invention, particularly suitable drugs include those that are relatively unstable when formulated as solid dosage forms, those that are adversely affected by the low pH conditions of the stomach, those that are adversely affected by exposure to enzymes in the gastrointestinal tract, and those that are desirable to provide to a patient via sustained or controlled release. Examples of suitable drugs include antiinflammatory drugs, both steroidal (e.g., hydrocortisone, prednisolone, triamcinolone) and nonsteroidal (e.g., naproxen, piroxicam); systemic antibacterials (e.g., erythromycin, tetracycline, gentamycin, sulfathiazole, nitrofurantoin, vancomycin, penicillins such as penicillin V, cephalosporins such as cephalexin, and quinolones such as norfloxacin, flumequine, ciprofloxacin, and ibafloxacin); antiprotazoals (e.g., metronidazole); antifungals (e.g., nystatin); coronary vasodilators; calcium channel blockers (e.g., nifedipine, diltiazem); bronchodilators (e.g., theophylline, pirbuterol, salmeterol, isoproterenol); enzyme inhibitors such as collagenase inhibitors, protease inhibitors, elastase inhibitors, lipoxygenase inhibitors, and angiotensin converting enzyme inhibitors (e.g., captopril, lisinopril); other antihypertensives (e.g., propranolol); leukotriene antagonists; anti-ulceratives such as H2 antagonists; steroidal hormones (e.g., progesterone, testosterone, estradiol); local anesthetics (e.g., lidocaine, benzocaine, propofol); cardiotonics (e.g., digitalis, digoxin); antitussives (e.g., codeine, dextromethorphan); antihistamines (e.g., diphenhydramine, chlorpheniramine, terfenadine); narcotic analgesics (e.g., morphine, fentanyl); peptide hormones (e.g., human or animal growth hormones, LHRH); cardioactive products such as atriopeptides; proteinaceous products (e.g., insulin); enzymes (e.g., anti-plaque enzymes, lysozyme, dextranase); antinauseants; anticonvulsants (e.g., carbamazine); immunosuppressives (e.g., cyclosporine); psychotherapeutics (e.g., diazepam); sedatives (e.g., phenobarbital); anticoagulants (e.g., heparin); analgesics (e.g., acetaminophen); antimigraine agents (e.g., ergotamine, melatonin, sumatripan); antiarrhythmic agents (e.g., flecainide); antiemetics (e.g., metoclopromide, ondansetron); anticancer agents (e.g., methotrexate); neurologic agents such as antidepressants (e.g., fluoxetine) and anti-anxiolytic drugs (e.g., paroxetine); hemostatics; and the like, as well as pharmaceutically acceptable salts and esters thereof. Proteins and peptides are particularly suitable for use with these chromonic compositions. Suitable examples include erythropoietins, interferons, insulin, monoclonal antibodies, blood factors, colony stimulating factors, growth hormones, interleukins, growth factors, therapeutic vaccines, and prophylactic vaccines. The amount of drug that constitutes a therapeutically effective amount can be readily determined by those skilled in the art with due consideration of the particular drug, the particular carrier, the particular dosing regimen, and the desired therapeutic effect. The amount of drug will typically vary from about 0.1 to about 70% by weight of the total weight of the water-insoluble chromonic matrix. In one aspect the drug is intercalated in the matrix.

The guest molecules encapsulated in the chromonic material can have any useful size or diameter. In many embodiments, these encapsulated guest molecules can have a mass mean or median diameter in a range from 25 to 1000 nanometers, or from 100 to 750 nanometers, or from 200 to 500 nanometers. In particular, these particle sizes may be desirable for oral delivery of drugs that are unstable in the intestine due to the presence of certain enzymes. Examples of such drugs include proteins, peptides, antibodies, and other biologic molecules that may be particularly sensitive to the body's enzymatic processes. In such cases, these small particles may be taken up into the intestinal wall directly, such that the particle primarily dissolves after passing the intestinal barrier, so that the amount of the sensitive drug exposed to the intestinal environment is minimized. Particles are typically spherical in their general shape, but may also take any other suitable shape, such as needles, cylinders, or plates.

These encapsulated guest molecules are dissolvable in an aqueous solution of univalent cations or other non-ionic compounds, such as surfactants. Typical univalent cations include sodium and potassium. The concentration of univalent cations needed to dissolve the encapsulated guest molecules will depend on the type and amount of the chromonic molecules within the matrix, but for complete dissolution of the encapsulated guest molecules there should generally be at least a molar amount of univalent cations equivalent to the molar amount of carboxy groups in the matrix. In this way, there will be at least one univalent cation to associate with each carboxy group.

The rate at which an encapsulated guest molecule dissolves may also be adjusted by adjusting the type and amount of multi-valent cation used for crosslinking. Although divalent cations will be sufficient to crosslink the matrix, higher valency cations will provide additional crosslinking and lead to slower dissolution rates. In addition to valency, dissolution rate will also depend on the particular cation type. For example, a non-coordinating divalent cation, such as magnesium, will generally lead to faster dissolution than a coordinating divalent cation, such as calcium or zinc, which has an empty electron orbital capable of forming a coordination bond with a free electron pair. Different cation types may be mixed so as to give an average cation valency that is not an integer. In particular, a mixture of divalent and trivalent cations will generally cause a slower dissolution rate than a like matrix where all of the cations are divalent. In one aspect, all of the guest molecules will be released over time, but it may be desired in certain applications to have only a portion of the guest molecules be released. For instance, the type or amount of chromonic molecule and multivalent cation may be adjusted such that the total amount of guest molecules that are released will vary depending on the environment into which they are placed. In one embodiment, the encapsulated guest molecules will not dissolve in an acidic solution, thus protecting acid sensitive guest molecules from degradation. In another, the encapsulated guest molecules will not dissolve in an acidic solution containing univalent cations, thus protecting acid sensitive guest molecules from degradation. In the particular instance where the guest molecule is a drug, two common types of general release profiles that are desired are immediate or sustained. For immediate release use it is typically desired that most of the drug will be released in a time period of less than about 4 hours, generally less than about 1 hour, often less than about 30 minutes, and in some cases less than about 10 minutes. In some instances it will desired that drug release will be nearly instantaneous, that is it will take place in a matter of seconds. For sustained (or controlled) release uses it is typically desired that most of the drug will be released over a time period greater than or equal to about 4 hours. Periods of one month or more may be desired, for example in various implantable applications. Oral sustained release dosages will generally release most of the drug over a time period of about 4 hours to about 14 days, sometimes about 12 hours to about 7 days. In one aspect it may be desired to release most of the drug over a time period of about 24 to about 48 hours. A combination of immediate and sustained release may also be desired, where for instance; a dosage provides an initial burst of release to rapidly alleviate a particular condition followed by a sustained delivery to provide extended treatment of the condition.

Chromonic material that encapsulates a drug finds particular use in oral dosage drug delivery. Typical oral dosage forms include solid dosages, such as tablets and capsules, but may also include other dosages administered orally, such as liquid suspensions and syrups. In one aspect, the encapsulated guest molecules will be particles that are stable in acidic solution and that will dissolve in an aqueous solution of univalent cations. In another aspect, the encapsulated guest molecules will be stable in the acidic environment of the stomach and will dissolve when passed into the non-acidic environment of the intestine when administered to an animal. When the encapsulated guest molecules are stable in acidic solution, the encapsulated guest molecules may generally be stable for periods of time longer than 1 hour, sometimes more than 12 hours, and may be stable for more than 24 hours when present in an acidic environment with a pH less than 7.0, for example less than about 5.0, and in some cases less than about 3.0.

In many embodiments, encapsulation of guest molecules using chromatic material optimally operates when the encapsulation solution has a chromatic material concentration in a range higher than 1 to 2% wt. It has been difficult to generate chromonic particles in the nanometer range using these higher chromonic concentrations. The process described below uses low chromonic concentrations to make chromonic particles in the nanometer range and then concentrates the chromonic material/solution after initiation of these particles. In some embodiments, this apparatus generates small droplets of chromonic material/solution using atomization technology, then dilutes the droplet stream with warm gas to heat the droplet stream, and then impinges the warm droplet stream with high velocity warm gas to break up agglomerations and provide effective heat transfer to evaporate the carrier fluid at a low temperature.

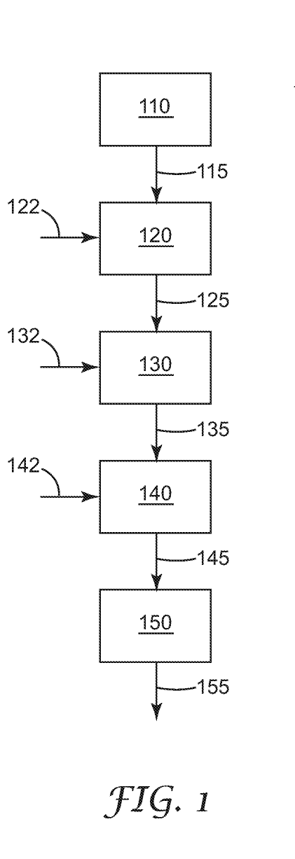

FIG. 1 is a schematic diagram of an illustrative apparatus 100 for forming the encapsulated guest molecules using chromonic nanoparticles. The chromonic nanoparticles are formed via atomization. Formation of the chromonic nanoparticles used to encapsulate the guest molecules is based upon the concept of atomizing a fluid or aerosol chromonic composition, which in many embodiments is substantially organic solvent-free, to form a plurality of fine liquid chromonic droplets. In many embodiments, the carrier fluid is water and the guest molecule is a drug. These chromonic droplets are contacted with a carrier or dilution gas, which causes the chromonic droplets to vaporize even at temperatures well below the boiling point of the chromonic droplets. Vaporization occurs quickly and completely, because the partial pressure of the vapor in admixture with the carrier gas is still well below the chromonic vapor's saturation pressure at the specified operating temperature. When the dilution or carrier gas, used in combination with atomization is heated, the gas provides the thermal and mechanical energy for vaporization. After vaporization, the chromonic nanoparticles can then be utilized to encapsulate or coat the guest molecule, as described below. In many embodiments, this process is maintained at a temperature below a temperature that is detrimental to the guest molecule.

The processes described herein may be practiced in a vacuum. Advantageously, however, atomization, vaporization, and encapsulation can occur at any desired pressure, including ambient pressure or above. Alternatively, or in addition, atomization, vaporization, and coating can occur at relatively low temperatures, so that temperature sensitive guest molecules or chromonic materials can be processed without degradation that might otherwise occur at higher temperatures.

Generally, atomization of the fluid chromonic coating composition can be accomplished using any atomization technique, including ultrasonic atomization, spinning disk atomization, and the like. In one embodiment, a first atomizer is a Model 9306 Six Jet Atomizer (TSI, Inc., Shoreview, Minn.), a second atomizer in is fluid connection to the outlet of the first atomizer and utilizes external gas-impingement technology in which atomization is achieved by impinging a conical-annular stream of gas into a central or axially flowing stream of gas with a stream of the fluid. This second atomizer is similar in design to the apparatus described in U.S. Pat. No. 6,045,864, incorporated herein by reference to the extent it does not conflict. The fluid may be an aerosol or a liquid. In some embodiments, the gas is heated, and the fluid stream flow is laminar at the time of collision, but this is not required. The energy of the collision breaks the fluid stream or aerosol composition into very fine droplets. The second atomizer reduces the size of the droplets even further than the first atomizer. Using this kind of collision to achieve atomization provides smaller atomized droplets with a narrower size distribution and a higher number density of droplets per volume (i.e., the droplets are smaller in the same overall volume) than can be achieved using some other atomization techniques. Additionally, the resultant droplets are almost immediately in intimate contact with the carrier gas, resulting in rapid, efficient vaporization.

As shown in FIG. 1, the illustrative apparatus 100 includes a solution reservoir 110 in fluid communication with a first atomizer 120. The solution reservoir 110 can be any useful size for holding an amount of solution mixture. One useful size is that which comes with the Model 9306 Six Jet Atomizer. In many embodiments, the solution mixture includes chromonic material, a guest molecule and a carrier fluid. In other embodiments, the solution mixture includes chromonic material and a carrier fluid and no guest molecule. The solution mixture is supplied 115 to the first atomizer 120 and is atomized by being collided/mixed with a first stream of dilution gas 122. The first dilution gas 122 can be any useful gas. In many embodiments, the first dilution gas 122 is an inert gas that can be heated to a specified temperature. In one embodiment, the first dilution gas 122 is dry nitrogen gas. The first atomizer 120 can include any number of inlet and outlet ports in any useful configuration. An illustrative example of a useful atomizer is a Model 9306 Six Jet Atomizer commercially available from TSI, Inc., Shoreview, Minn.

The first atomizer 120 produces a particle stream contained within a flow passage, which is termed herein as a ‘pre-atomized’ particle stream 125. The pre-atomized particle stream 125 can have a mean particle diameter of any useful or desired size that is produced by varying the operating conditions of the first atomizer 120. In many embodiments, this pre-atomized particle stream 125 has a mean particle size in a range from 1 to 100 micrometers, or from 1 to 50 micrometers, or from 1 to 20 micrometers, or from 1 to 10 micrometers, or from 1 to 5 micrometers, as desired. In one embodiment, this pre-atomized particle stream 125 includes chromonic material, a guest molecule, a carrier fluid, and the first dilution gas. In another embodiment, this pre-atomized particle stream 125 includes chromonic material, a carrier fluid, and the first dilution gas, and not the guest molecule.

The pre-atomized particle stream 125 is then fed into a second atomizer 130 to further atomize the pre-atomized particle stream 125 and form an atomized particle stream 135 by being collided with a second stream of dilution gas 132. The second stream of gas 132 can be any useful gas that is the same or different than the first dilution gas 122. In many embodiments, the second dilution gas 132 is an inert gas that can be heated to a specified temperature. In one embodiment, the second dilution gas 132 is dry nitrogen gas. The second atomizer 130 can include any number of inlet and outlet ports in any useful configuration. An illustrative example of a useful second atomizer is described in U.S. Pat. No. 6,045,864, which in incorporated by reference herein to the extent it does not conflict with this disclosure. In one illustrative embodiment, the second atomizer is similar to the atomizer described in U.S. Pat. No. 6,045,864, however the axial flow channel is completely open from inlet to outlet and the annular slot can be varied in the axial dimension by shimming the flange plates instead of thread adjustment. By opening the slot, a higher volume of gas can flow to impinge upon the pre-atomized particle stream thereby changing the mechanical energy transfer rate from the second atomizer dilution gas to the atomized particle stream. This second atomizer can cause significant turbulence in the atomized particle stream thereby adding to the mixing effect of the heated gas and the particle stream.

In some embodiments, a third dilution gas (not shown) can be introduced between the first atomizer 120 and the second atomizer 130, to add dilution and thermal heat transfer capability to the system 100. This third dilution gas can be introduced counter-currently to the particle stream flow to induce additional mixing and randomization of the pre-atomized stream 125.

The atomized particle stream 135 can have a mean particle diameter of any useful or desired size that is produced by varying the operating conditions of the second atomizer 130. In many embodiments, this atomized particle stream 135 has a mean particle size in a range from 50 to 1000 nanometers, or from 50 to 900 nanometers, or from 50 to 750 nanometers, or from 100 to 500 nanometers, as desired. In one embodiment, this atomized particle stream 135 includes chromonic material, a guest molecule, a carrier fluid, and the second dilution gas. In another embodiment, this atomized particle stream 135 includes chromonic material, a carrier fluid, and the second dilution gas, and not the guest molecule.

A drying or evaporation chamber 140 can be in fluid communication with the second atomizer 130. The atomized particle stream 135 can be supplied to the drying or evaporation chamber 140 from the second atomizer 130. At least a portion of the carrier fluid is evaporated by the drying or evaporation chamber 140 to form a concentrated atomized particle stream 145. The concentrated atomized particle stream 145 can have a mean particle diameter of any useful or desired size that is produced by varying the operating conditions of the drying or evaporation chamber 140 (as well as any upstream operating parameter). In many embodiments, this concentrated atomized particle stream 145 has a mean particle size in a range from 25 to 750 nanometers, or from 25 to 500 nanometers, or from 25 to 500 nanometers, or from 25 to 250 nanometers, as desired. In one embodiment, this concentrated atomized particle stream 145 includes chromonic material, a guest molecule, and the drying gas. In another embodiment, this concentrated atomized particle stream 145 includes chromonic material and the drying gas, and not the guest molecule.

Heat can be provided to the drying or evaporation chamber 140 via external heating and/or a drying gas stream 142 at a specified temperature. The drying gas 142 can be any useful gas that is the same or different than the first dilution gas 122 and/or the second dilution gas 132. In many embodiments, the drying gas 142 is an inert gas that can be heated to a specified temperature. In one embodiment, the drying gas 142 is dry nitrogen gas. In many embodiments, at least 50% of the carrier fluid is evaporated from the atomized particle stream 135 to form the concentrated atomized particle stream 145. In other embodiments, at least 75% of the carrier fluid is evaporated from the atomized particle stream 135 to form the concentrated atomized particle stream 145. In other embodiments, at least 90% of the carrier fluid is evaporated from the atomized particle stream 135 to form the concentrated atomized particle stream 145.

The concentrated atomized particle stream 145 can then be provide for further processing, as desired. In some embodiments, the concentrated particle stream 145 is provided to an encapsulation chamber 150 to form encapsulated guest molecules 155. In many embodiments, these encapsulated guest molecules 155 are formed via cross-linking the chromonic nanoparticles with a multi-valent cation such as, for example CaCl₂ or ZnCl₂.

The encapsulated guest molecules 155 can have a mean particle diameter of any useful or desired size that is produced by varying the operating conditions of the coating chamber 150. In many embodiments, these encapsulated guest molecules 155 have a mean particle size in a range from 25 to 1000 nanometers, or from 75 to 750 nanometers, or from 100 to 750 nanometers, or from 100 to 500 nanometers, or from 200 to 500 nanometers, as desired. In one embodiment, these encapsulated guest molecules 155 include chromonic material disposed about the guest molecule and carrier fluid. In another embodiment, these encapsulated guest molecules 155 include chromonic material disposed about the guest molecule and not the carrier fluid.

In one illustrative embodiment, all of the processing steps occur within a five degree Celsius temperature range. In another illustrative embodiment, all of the processing steps occur at a temperature of less than 40° Celsius. In a further embodiment, all of the processing steps occur within a five degree Celsius temperature range that is below 40° Celsius.

The present invention should not be considered limited to the particular examples described herein, but rather should be understood to cover all aspects of the invention as fairly set out in the attached claims. Various modifications, equivalent processes, as well as numerous structures to which the present invention can be applicable will be readily apparent to those of skill in the art to which the present invention is directed upon review of the instant specification.

### EXAMPLES

Unless otherwise noted, all reagents and solvents were or can be obtained from Sigma-Aldrich Co., St. Louis, Mo.

As used herein,

“purified water” refers to water available under the trade designation “OMNISOLVE” from EMD Chemicals, Inc., Gibbstown, N.J.;

“INNOVATOL PD60” refers to polyalditol, obtained from Innova LLC (Muscatine, Iowa).

### Preparative Example 1

Preparation of Oleyl Phosphonic Acid

A mixture of 60.0 g (0.209 mol) of oleyl chloride (obtained from TCI America, Portland, Oreg.) and 84.2 g (0.525 mol) of triethyl phosphite (obtained from Alfa Aesar, Ward Hill, Mass.) was stirred and heated at 150° C. After 2 days, an additional 87.0 g (0.524 mol) of triethyl phosphite was added, and heating was continued. After an additional 6 days, an additional 87.0 g (0.524 mol) of triethyl phosphite was added, and the reaction temperature was raised to 170° C. After 14 days more, the mixture was distilled under reduced pressure, and then bulb-to-bulb distillation of the concentrated mixture afforded 78.3 g of oleyl diethyl phosphonate as a clear, colorless liquid (b.p. 170-185° C. at 4 Pa (0.03 mm Hg)). To a solution of 48.6 g (0.125 mol) of oleyl diethyl phosphonate in 150 mL of dichloromethane there was added dropwise with stirring 42.1 g (0.275 mol) of bromotrimethylsilane. After 24 hours at room temperature, the solution was concentrated using a rotary evaporator, and then the resultant mixture was dissolved in 250 mL of methanol. This solution was stirred at room temperature for 1 hour and then was concentrated using a rotary evaporator. Dissolution in methanol and concentration were repeated two times. The resultant product was dissolved in 500 mL of hexane, and this solution was filtered. The filtrate was chilled in dry ice and then the precipitated solid was collected by filtration and was washed with cold hexane, providing 28.6 g of oleyl phosphonic acid as a white solid. The ¹H, ¹³C, and ³¹P NMR spectra of the product were consistent with the assigned structure.

### Example 1

Preparation of Chromonic Nanoparticles Including Insulin

An aqueous mixture of bovine insulin was prepared by stirring together a mixture of purified water (8.0 g), ethanolamine (0.023 g), oleyl phosphonic acid (0.1 g of a 10 weight percent aqueous solution), and insulin from bovine pancreas (0.1 g). This mixture was magnetically stirred for ten minutes, and then the chromonic compound of Formula I (in which R₃ was the methylimidazolium group of Formula V) (1.0 g) was added to the mixture, followed by ethanolamine (0.092 g). This solution was diluted ten times with purified water to afford an approximately 1 weight percent chromonic insulin solution.

Separately, a crosslinking solution was prepared by stirring together purified water (7.5 g), calcium chloride (0.995 g), zinc chloride (0.005 g), and Innovatol PD 60 (1.5 g). This crosslinking solution was coated on glass film and exposed to the impinging concentrated chromonic nanoparticles produced by the apparatus described below.

The nanoparticle generating apparatus included a solution reservoir in fluid communication with a first atomizer. A second atomizer was in fluid communication with the first atomizer and a drying or evaporation chamber was in fluid communication with the second atomizer. The glass film with the cross-linking coating was in fluid communication with the second atomizer through the drying or evaporation chamber.

The first atomizer was a Six Jet Atomizer (P/N 1990143) Model 9306 (available from TSI, Inc., Shoreview, Minn.). The second atomizer was similar to the atomizer described in U.S. Pat. No. 6,045,864, however the axial flow channel was completely open from inlet to outlet and the annular slot was varied in the axial dimension by shimming the flange plates. Dry nitrogen gas was used as the dilution gas for both atomizers, the evaporation chamber and as dilution gas between the atomizers at varying flow rates. The dry nitrogen gas and entire process was maintained at a temperature in a range from 35 to 40 degrees centigrade.

As stated above, the 1 weight percent chromonic insulin solution was placed in the solution reservoir and fed to the first atomizer. The first atomizer was operated such that both visible droplets (approximately one micrometer or larger) and droplets that could not be seen with the unaided eye (less than approximately one micrometer) were produced by the first atomizer. These droplets (the pre-atomized particles stream) were then fed into the second atomizer to form an atomized particles stream (particles having a mean particle size of less than approximately one micrometer). This atomized particle stream then flowed into an evaporation chamber and additional dry nitrogen dilution gas was mixed therewith to form a concentrated nanoparticle stream. This concentrated nanoparticle stream then impinged upon the glass film (on which the crosslinking solution had been coated) to form chromonic encapsulated insulin nanoparticles. The product was analyzed by transmission electron microscopy and the chromonic encapsulated insulin nanoparticles were found to have a mean particle size range of approximately 25 to approximately 100 nanometers.

The temperature and flow settings for the apparatus shown in FIG. 1 (used to produce this example) were adjusted based on the ideal operating temperature, of about 38 degrees centigrade. The dilution and mixing gas temperatures were held at 40 to 45 degrees centigrade and the total flow between the two were about 15-20 liters per minute. The inlet to the atomizers on the six-jet system had a setpoint of 60 degrees centigrade at an inlet pressure of about 410 KPa (60 psi) (estimated 20 liters per minute flow). The impingement nozzle (atomization gas) was set to 90 degrees centigrade, expansion losses required these higher temperature settings, and the flow was set to about 15-20 liters per minute. Total gas flow was estimated to be 60 liters per minute for proper dilution and vaporization for the estimated solvent (water) thermodynamic properties.

The present invention has been described with reference to several embodiments thereof. The foregoing detailed description and examples have been provided for clarity of understanding only, and no unnecessary limitations are to be understood therefrom. It will be apparent to those skilled in the art that many changes can be made to the described embodiments without departing from the spirit and scope of the invention. Thus, the scope of the invention should not be limited to the exact details of the compositions and structures described herein, but rather by the language of the claims that follow. The complete disclosures of the patents, patent documents and publications cited herein are incorporated by reference in their entirety as if each were individually incorporated. In case of any conflict, the present specification, including definitions, shall control.

We claim:

1. A method comprising:
atomizing a solution to form a pre-atomized particle stream, the solution comprising chromonic material, a guest molecule and a carrier fluid;
atomizing the pre-atomized particle stream to form an atomized particle stream;
evaporating at least a portion of the carrier fluid from the atomized particle stream to form a concentrated atomized particle stream; and
forming encapsulated guest molecules from the concentrated atomized particle stream, the guest molecules encapsulated in the chromonic material.

2. A method according to claim 1, wherein the atomizing a solution step comprises atomizing a solution to form a pre-atomized particle stream wherein the pre-atomized particle stream has a mean particle size in a range from 1 to 20 micrometers.

3. A method according to claim 1, wherein the atomizing the pre-atomized particle stream step comprises atomizing the pre-atomized particle stream to form an atomized particle stream wherein the atomized particle stream has a mean particle size in a range from 50 to 1000 nanometers.

4. A method according to claim 1, wherein the evaporating step comprises evaporating carrier fluid from the atomized particle stream to form a concentrated atomized particle stream wherein the concentrated atomized particle stream has a mean particle size in a range from 25 to 500 nanometers.

5. A method according to claim 1, wherein the forming encapsulated guest molecules step comprises forming encapsulated guest molecules from the concentrated atomized particle stream, the guest molecules encapsulated in the chromonic material and the encapsulated guest molecules have a mean size in a range from 25 to 1000 nanometers.

6. A method according to claim 1, wherein the forming encapsulated guest molecules step comprises forming encapsulated guest molecules from the concentrated atomized particle stream, the guest molecules encapsulated in the chromonic material and the encapsulated guest molecules have a mean size in a range from 200 to 500 nanometers.

7. A method according to claim 1, wherein the atomizing steps, evaporating step, and forming step occur within a five degree Celsius temperature range.

8. A method according to claim 1, wherein the atomizing steps, evaporating step, and forming step occur at less than 40° Celsius.

9. A method according to claim 1, further comprising diluting the pre-atomized particle stream with a gas.

10. A method according to claim 1, further comprising diluting the atomized particle stream with a gas.

11. A method according to claim 1, wherein the forming encapsulated guest molecules step further comprises cross-linking non-covalently the chromonic material to form encapsulated guest molecules from the concentrated atomized particle stream, the guest molecules encapsulated in the chromonic material.

12. A method according to claim 1, wherein the chromonic material exhibits a chromonic M phase during the atomizing steps, evaporating step, and forming step.

13. A method according to claim 1, wherein the atomizing a solution to form a pre-atomized particle stream comprises, atomizing a solution to form a pre-atomized particle stream, the solution comprising chromonic material, a guest molecule and a carrier fluid, wherein the guest molecule comprises insulin.

14. A method according to claim 1, wherein the atomizing a solution to form a pre-atomized particle stream comprises, atomizing a solution to form a pre-atomized particle stream, the solution comprising a 0.01% wt to 2% wt chromonic material/guest molecule.

15. An apparatus comprising;
a solution reservoir in fluid communication with a first atomizer, the solution reservoir containing chromonic material, a guest molecule and a carrier fluid;
a second atomizer in fluid communication with the first atomizer; and
a drying chamber in fluid communication with the second atomizer.

16. An apparatus according to claim 15, further comprising a first dilution gas inlet in fluid communication with the first atomizer and a second dilution gas inlet in fluid communication with the second atomizer.

17. An apparatus according to claim 16, further comprising a first dilution gas heater in fluid communication with the first dilution gas inlet and a second dilution gas heater in fluid communication with the second dilution gas inlet.

18. An apparatus according to claim 15, further comprising an encapsulation chamber in fluid communication with the drying chamber, the encapsulation chamber containing multivalent cations.

19. An apparatus according to claim 15, wherein the guest molecule comprises insulin.
